(19) United States
(12) Patent Application Publication
(10) Pub. No.: US 2024/0237967 A1
(43) Pub. Date: Jul. 18, 2024

(54) DEVICES, SYSTEMS, AND METHODS FOR EXTENDING AN ENDOSCOPE

(71) Applicant: EndoSound, Inc., Portland, OR (US)

(72) Inventors: Stephen E. Steinberg, Boca Raton, FL (US); Scott Sutherland Corbett, III, Portland, OR (US); William J. McDonough, Portland, OR (US)

(73) Assignee: EndoSound, Inc., Portland, OR (US)

(21) Appl. No.: 18/562,303

(22) PCT Filed: May 18, 2022
(86) PCT No.: PCT/US2022/072408

§ 371 Date: Nov. 18, 2023

Related U.S. Application Data
(60) Provisional application No. 63/189,933, filed on May 18, 2021.

(51) Int. Cl. A61B 8/00; A61B 8/08; A61B 8/12; A61B 10/04; A61B 17/34; A61M 25/00

(52) U.S. Cl. CPC A61B 8/445; A61B 8/08; A61B 8/12; A61B 10/04; A61B 17/3403; A61B 2010/045; A61B 2017/3413; A61M 2025/0095

(57) ABSTRACT

An endoscope assembly is disclosed herein. The endoscope assembly can include an endoscope, having a proximal and a distal end, and defining a lumen, and having a proximal lumen port, permitting entry of a lengthwise needle or other device into said lumen; and a tubular extension, having a proximal and distal end, and having said distal end affixed to said proximal lumen port, an ultrasound endoscope needle assembly attached to proximal end of said tubular extension and having: a proximal handle; and a needle having a distal end, extending distally out of said handle; and wherein said needle fits through said tubular extension, and through said proximal lumen port into said lumen, and with said needle positioned so that said distal end is within said lumen but can be extended out of said lumen by manipulation of said handle.

### CROSS-REFERENCE TO RELATED APPLICATIONS

This application claims the benefit of and priority under 35 U.S.C. § 119(e) to U.S. Provisional Patent Application No. 63/189,933, filed May 18, 2021, entitled EXTENSION FOR AN ENDOSCOPE, the disclosure of which is hereby incorporated by reference in its entirety.

### FIELD

The present disclosure is generally related to the field of endoscopy, including the use of optical scopes (e.g., endoscopes, gastroscopes, duodenoscopes, etc.) to perform ultrasound-assisted biopsies, including the use of needles, in conjunction with ultrasound systems.

### BACKGROUND

An endoscopic ultrasound system (EUS), and in particular the linear version, has the capability of visualizing structures adjacent to the lumen of the esophagus, stomach, or intestine in which the scope is position. It does so by creating a sound image of nearby structures. The linear EUS scope (that is, the endoscope portion of the EUS, which also includes an imaging station) has a channel that allows a variety of tools (e.g., needles) to be passed by the operator through entry point of the channel and down the entire length of the scope, exiting at the end, which is in the body and further, in the field of view of the ultrasound. This allows therapeutic maneuvers (e.g., needle biopsy) to be performed under ultrasound visualization. Available therapeutic devices are designed to a length that allows them to be firmly attached to the channel entry point and to be positioned appropriately at the distal end of the endoscope.

A conventional gastroscope is a device designed to be extended through the esophagus to the stomach, whereby way of a miniaturized digital camera, a view of the stomach interior may be obtained. A specimen may be taken from the stomach interior, by way of a device that has a sharp pair of jaws that can be introduced through a lumen in the gastroscope, bite into the stomach lining, and then be withdrawn through the lumen to provide the medical team with a specimen for further analysis. Other maneuvers and manipulations may be performed as well.

In order to safely perform a needle biopsy, however, the operator would have to have the ability view the target site, optically hidden beneath a layer of tissue, in order to avoid risking the puncture of an interior membrane. As conventional gastroscopes are entirely optical, this capability is absent. Accordingly, needles sized for gastroscopes are not currently available for various procedures (e.g., fine-needle aspirations, fine-needle biopsies, etc.). Such a needle would be sized to enter the gastroscope lumen at the proximal lumen port and extend out of the lumen at the distal end, without having an awkward length remaining extending out of the proximal lumen port when the distal tip of the needle is just at the distal end of the gastroscope. The needles that are currently available for use with endoscopic ultrasound systems (EUS) are too long to be used with a gastroscope.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, an endoscope assembly is disclosed. The endoscope assembly can include an endoscope, having a proximal and a distal end, and defining a lumen, and having a proximal lumen port, permitting entry of a lengthwise needle or other device into said lumen; and a tubular extension, having a proximal and distal end, and having said distal end affixed to said proximal lumen port, an ultrasound endoscope needle assembly attached to proximal end of said tubular extension and having: a proximal handle; and a needle having a distal end, extending distally out of said handle; and wherein said needle fits through said tubular extension, and through said proximal lumen port into said lumen, and with said needle positioned so that said distal end is within said lumen but can be extended out of said lumen by manipulation of said handle.

In various aspects, a method of retrofitting an endoscope is disclosed. The endoscope can include a proximal and distal end, and can define a lumen, and can have a proximal lumen port, to accept and support an ultrasound endoscope needle assembly. The endoscope can further include a handle that permits the extension and retraction of said needle, over a range. The method can include providing a tubular extension having a proximal and a distal end, and affixing said distal end of said tubular extension to said proximal lumen port, wherein said tubular extension is sized to permit said ultrasound endoscope needle assembly when affixed to said proximal end of said extension to extend through said extension and said lumen so that said distal tip of said needle can be withdrawn into said lumen and extended out of said lumen by manipulating said handle.

In various aspects, a tubular extension for a make of gastroscope is disclosed. The tubular extensions can include a proximal and distal end, and wherein said distal end has a receptive connector for an endoscope proximal lumen port insertive connector for said make of gastroscope and wherein said proximal end has an endoscope proximal lumen port insertive connector.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF DRAWINGS

Various embodiments of the invention are disclosed in the following detailed description and accompanying drawings.

FIG. 1 illustrates an isometric view of an imaging assembly having an endoscope and an ultrasound imaging assembly added on, according to at least one non-limiting aspect of the present disclosure;

FIG. 2 illustrates an isometric view of the distal end of the assembly of FIG. 1;

FIG. 3 illustrates a side view of the distal end of the assembly of FIG. 1, in a first position;

FIG. 4 illustrates a side view of the distal end of the assembly of FIG. 1, in a second position;

FIG. 5 illustrates a side view of the distal end of an alternative embodiment of an imaging assembly, according to at least one non-limiting aspect of the present disclosure;

FIG. 6 illustrates a side view of another alternative embodiment of an imaging assembly, according to at least one non-limiting aspect of the present disclosure;

FIG. 7 illustrates an alternative embodiment of an imaging assembly, having an ultrasound imaging sub-assembly that includes an ultrasound head movement assembly that is detachable and three alternative needle guides, according to at least one non-limiting aspect of the present disclosure;

FIG. 8 illustrates a sectional view of a of the assembly of FIG. 7, showing a needle guide in a deployed state;

FIG. 9 illustrates a detail view of a portion of the assembly of FIG. 7, showing two of the parts disassembled from each other;

FIG. 10 illustrates the detail view of FIG. 9 but showing the two parts joined;

FIG. 11 illustrates a sectional view taken along line 11-11, of FIG. 10;

FIG. 12 illustrates a sectional view taken along line 12-12 of FIG. 10;

FIG. 13 illustrates a sectional view of the tip of the assembly of FIG. 7, showing the needle guide used to guide a needle;

FIG. 14 illustrates a sectional partial view of the assembly of FIG. 7 having a different style of needle guide′

FIG. 15 illustrates a view of the assembly of FIG. 14, showing the needle guide in use;

FIG. 16 illustrates an isometric view of a duodenoscope assembly, having an ultrasound imaging sub-assembly, according to at least one non-limiting aspect of the present disclosure;

FIG. 17 illustrates an isometric view of the assembly of FIG. 16, in a disassembled state;

FIG. 18 illustrates a sectional view of the assembly of FIG. 16, showing a different position for a portion of the assembly, in dashed line;

FIG. 19 illustrates an isometric view of an alternative embodiment of a duodenoscope assembly, according to at least one non-limiting aspect of the present disclosure;

FIG. 20 illustrates an isometric of the assembly of FIG. 19, in a disassembled state;

FIG. 21 shows an isometric view of a prior art assembly including an endoscope and a needle assembly;

FIG. 22 shows an isometric view of an extender attached to the proximal lumen port of an endoscope, according to at least one non-limiting aspect of the present disclosure;

FIG. 23 shows an isometric view of an extender attached to the proximal lumen port of an endoscope, and having a further unit attached to its distal end;

FIG. 24 illustrates a side view of the extender shown in FIG. 22;

FIG. 25 illustrates a sectional view of the extender shown in FIG. 22, presented in a collapsed configuration and at a larger scale, than shown in FIG. 24; and

FIG. 26 illustrates a flow chart of a method of adjusting the length of a needle using the extender of FIG. 22, in accordance with at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION AND EMBODIMENTS

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

To assist the description of the scope and its components the coordinate terms “proximal” and “distal” are used to describe the disclosed embodiments. The terms are used consistently with the description of the exemplary applications and are in reference to the body of a patient, while the gastroscope, which is an essential part of the invention or its environment is in use, having been introduced into a patient's esophagus. In other words, proximal components are outside the body, or closer to the surface, while distal components are in the body, or further in the body, compared to a more proximal component.

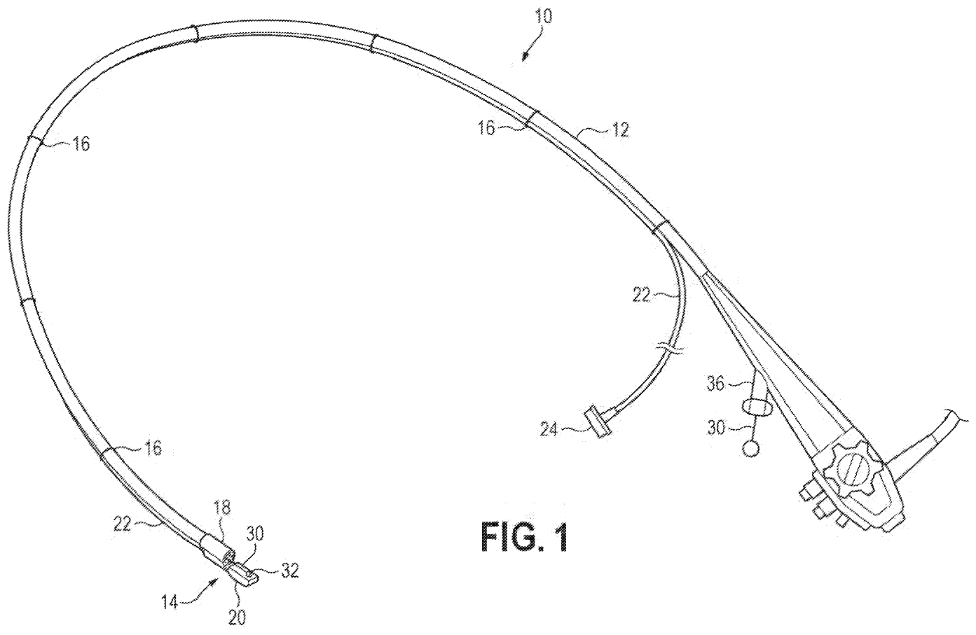

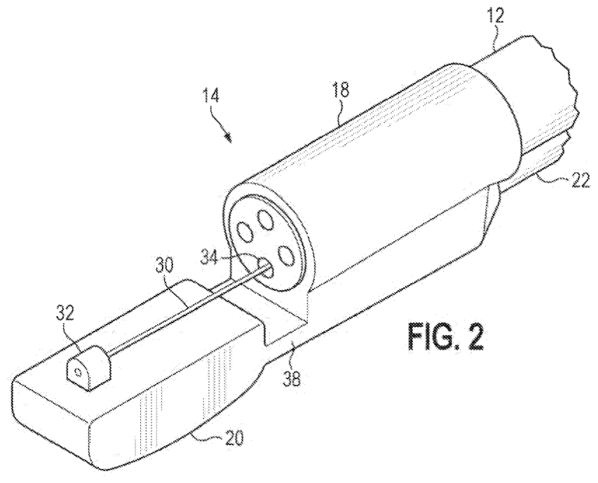

In a first preferred embodiment, an imaging assembly 10 includes an upper endoscope 12 and an ultrasound assembly 14 that has been attached to endoscope 12 by means of retaining element 18, integral to ultrasound assembly 14. In an alternative preferred embodiment, a retaining element is provided that is separate from ultrasound assembly 14 but works cooperatively to retain assembly 14 on endoscope 12. Assembly 14 also includes an ultrasound imaging (also referred to as “transducer”) head 20 that is electrically connected to a multiple signal pathway cable 22 by way of a flex circuit 50 (which is also a form of a signal pathway cable), that includes a set of parallel electrical leads, which may be traces. Cable 22, which has a multiplicity of signal pathways extending therethrough terminates in a connector 24, adapted to connect to an imaging station. Elements 16, which may be rubber bands, or some other form of elastic bands or clips, help to retain cable 22, to the side of endoscope 12. A tension member 30, such as a wire (which may also have some compressive strength) is attached to a fixation point 32 on ultrasound imaging head 20 and extends through a lumen 34 (FIG. 2) to emerge outside of a port 36 on the proximal end of endoscope 12, to be manipulated. In embodiments, tension member 30, does not extend through lumen 34, but extends along the side of endoscope 12, and in some embodiments is retained by elements 16, which are modified from the simple shapes shown in FIG. 1, to include eyelets, to create a guide path for tension member 30. In one embodiment, tension member 30 is connected to controls on the proximal end of endoscope 12, to facilitate manipulation. In embodiments, these controls may take the form of a spool, that can be easily let out, or drawn in. Endoscope 12 also is equipped with intrinsic controls for deflecting the tip of the insertion tube, to facilitate introduction to a site of interest.

In an alternative embodiment, tension member 30 is replaced by a tension member extending along the exterior of the endoscope, to a fixation point on the end of the endoscope. A physician may exert traction or pulsion on tension member 30 in any one of a variety of ways, to cause ultrasound imaging head 20 to bend forward or back toward retaining element 18, as permitted by a resiliently flexible neck 38 (FIG. 2). In one method a rotatable element is used to draw in tension member 30 or push it out.

In preferred embodiment endoscope 12 includes an element at its distal end to guide the alignment of the retaining element 18. For example, endoscope 12 many include a groove at its distal end, into which a key element on retaining element 18 engages. In another embodiment, an orientation guide includes a peg that fits into the lumen 34 and is used to guide the correct orientation of retaining element 18. In one embodiment, assembly 14 is made for intended disposal, after a single use, and is used in this manner. In another embodiment, assembly 14 is constructed so as to be prepared and/or cleaned appropriately for reuse, after use, and then reused. Although until recently generally disinfection procedures were deemed adequate, the detection of instances of the spread of infection through endoscope has given rise to the use of high-end disinfection techniques for endoscopes. These disinfection techniques make use of chemicals to kill any pathogens left on the scope after use. Other disinfection or sterilization techniques may be used, including processing using of UV light and/or a gas, such as ozone. In the context of this application, the term “cleaning” encompasses all disinfection and sterilization techniques. Generally, the materials used in endoscopes are such that autoclaving an endoscope, or an attachment thereto is not feasible.

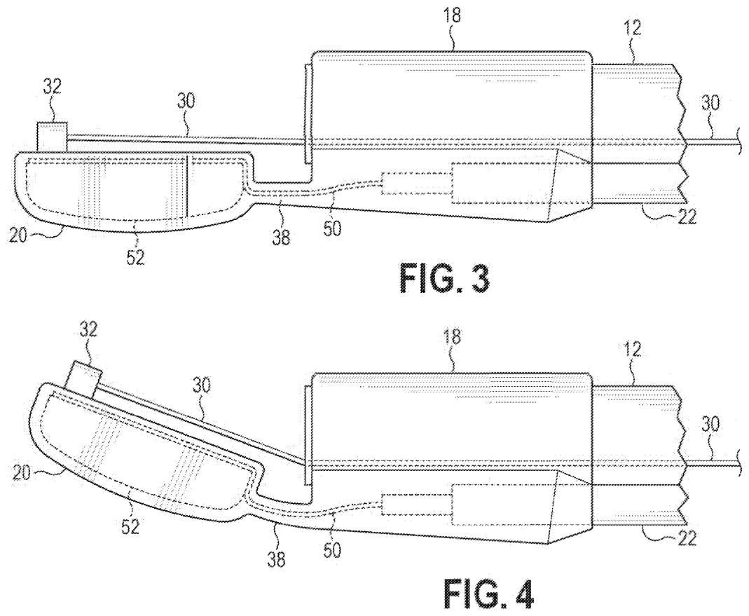

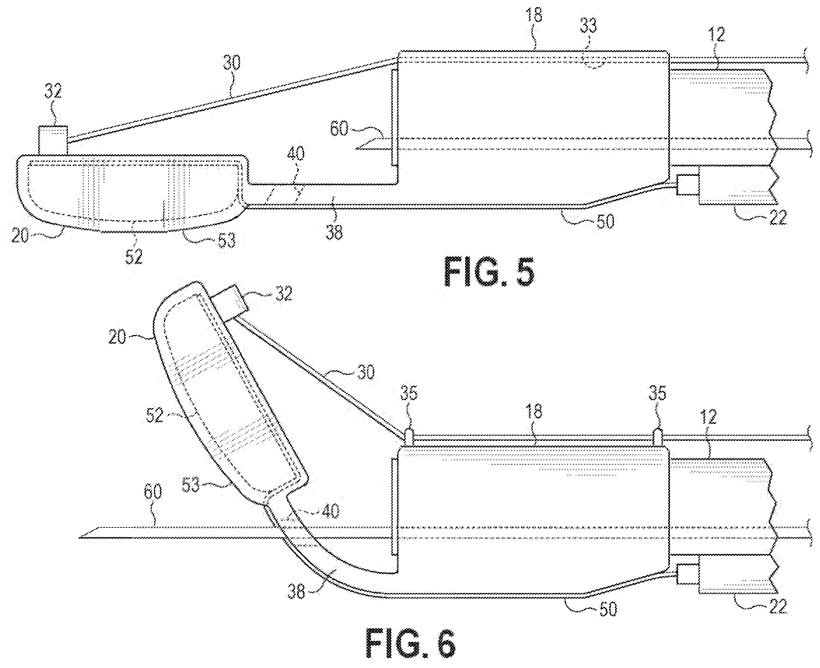

Referring to FIGS. 3 and 4, a flex circuit 50, which passes through the flexible neck 38, electrically connects imaging head 20 to cable 22. Flex circuit 50 has an electrical lead for each transducer element in an ultrasound element array 52, resident in the ultrasound imaging head 20, to drive ultrasound element array 52 and relay signals from it. Array 52 is covered with a protective coating 53 (FIGS. 5 and 6). In an alternative preferred embodiment, flex circuit 50 extends from imaging head 20 to (or through) connector 24, may define a plurality of coax cables and may directly contact the elements of the ultrasound array 52. In another alternative embodiment, cable 22 includes a set of coax cable bound together with an adherent and protective substance, such as a polymer, and extends from connector 24 to imaging head 20. In yet another embodiment, a fiber optic cable is used in place of cable 22, with light to electric convertors at its distal end. In any one of these arrangements elements 22 and 50 could be termed separately or in combination as a multiple signal pathway cable.

In a preferred embodiment, a biopsy needle 60 (FIGS. 5 and 6), which forms the sharpened, distal portion of a long, flexible, hollow-core wire, is provided. This wire is sheathed in a flexible conduit (not shown), thin enough to extend through the lumen 34 and protecting endoscope 12 from being damaged by needle 60. Once the conduit reaches the distal end of endoscope 12, it may be pushed out to extend from lumen 34, and provide further guidance for needle 60, which is pushed out of the conduit at a point distal to the end of endoscope 12. Alternatively, the conduit may be pushed roughly to the end of lumen 34, with the needle 60 pushed out of the conduit at that point.

Referring to FIG. 5, in an alternative preferred embodiment tension member 30 extends through a channel 33 in retaining element 18 to reach fixation point 32. This figure also shows a needle 60 that has been pushed through a lumen of the endoscope 12 and is emerging from the distal end of the lumen. An aperture 40 is defined in neck 38, corresponding to an aperture in flex circuit 50, aligned with aperture 40. FIG. 6 shows an embodiment that is similar to that of FIG. 5, but instead with tension member 30 extending through a pair of eyelets 35, supported on the retaining element 18. As well as showing a slightly different embodiment. FIG. 6 also shows imaging head 20 retracted and needle 60 extending through aperture 40, as it would be in order to take a biopsy. Notably, in this position the needle would be within the field of view of ultrasound array 52. In embodiments, tension member 30 can pull head 20 into an obtuse angle, relative to the distal end of the endoscope 12. Generally, aperture 40 is in the shape of a long oval, so that the needle 60 can pass through it over a long range of degree of bending of neck 38. In another preferred embodiment, the flexible conduit is extended distally from lumen 34 into a v-shaped indentation (not shown) on surface of flexible neck 38, aligning the conduit so that the needle 60 is aligned to pass through aperture 40.

To use imaging assembly 10, ultrasound assembly 14 is attached to endoscope 12 by means of retaining element 18. In an alternative embodiment, rubber bands or clips 16 (FIG. 1) retain cable 22 to the side of endoscope 12. Imaging head 20 is then delivered to an area of interest, by means of standard endoscope introduction techniques. Imaging head 20 may then be moved to gain imagery of the area of interest by dedicated controls which control the ultrasound imaging head 20 deflection. If there appears to be a finding to be sampled, needle 60 may be introduced through an endoscope lumen and through aperture 40 and used to take a biopsy, inject a drug, or otherwise effect a medical procedure. Finally, needle 60 is retracted through the lumen of endoscope 12 and the endoscope is retrieved from the patient's body. In other embodiments, needle 60 is not included and an assembly that is similar to imaging assembly 10 but without needle 60 and related elements, is used for imaging alone, or for introduction of some other device.

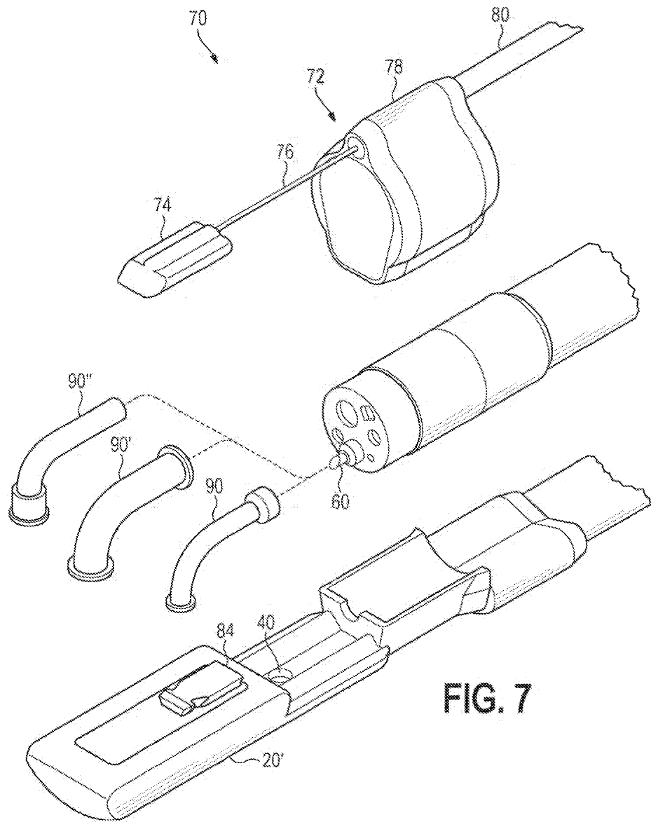

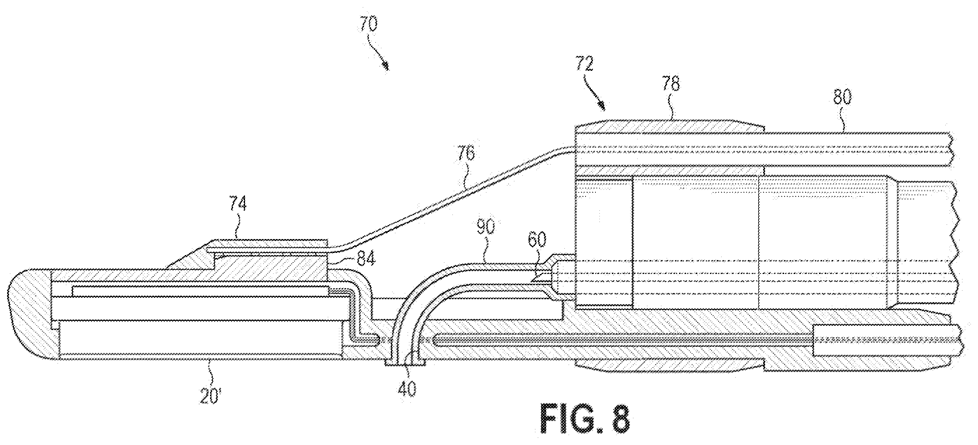

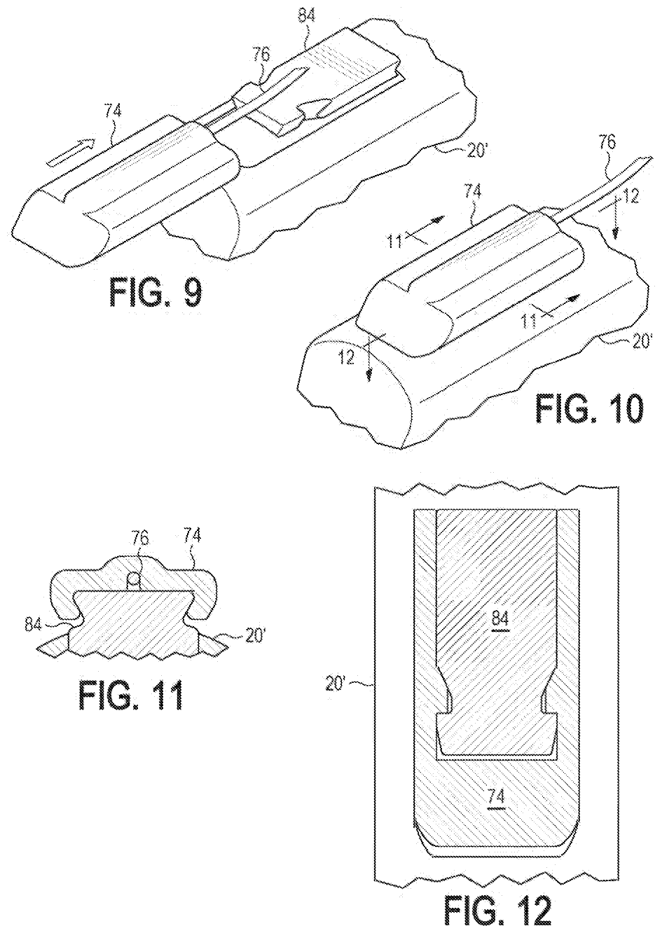

FIGS. 7-13 show an alternative embodiment 70 of the assembly 10, with the further innovation of a disposable head-movement sub-assembly 72, which includes a head clip 74, a movement cable 76, a cable clip 78 and a conduit 80, holding the major portion of movement cable 76. A clip-hold 84 is defined on the back of imaging head 20′. Further, the cable clip 78 holds imaging head and other portions of the ultrasound assembly 14′, including communicative cable 22′, to the endoscope 12. FIGS. 9-12 show engagement of head clip 74 to clip-hold 84. FIG. 9 shows head clip 74 distal to and being pulled back onto clip-hold 84, with FIG. 10 showing head clip 74 engaged to clip-hold 84 and FIGS. 11 and 12 showing different sectional views of head clip 74 and clip-hold 84 engaged together.

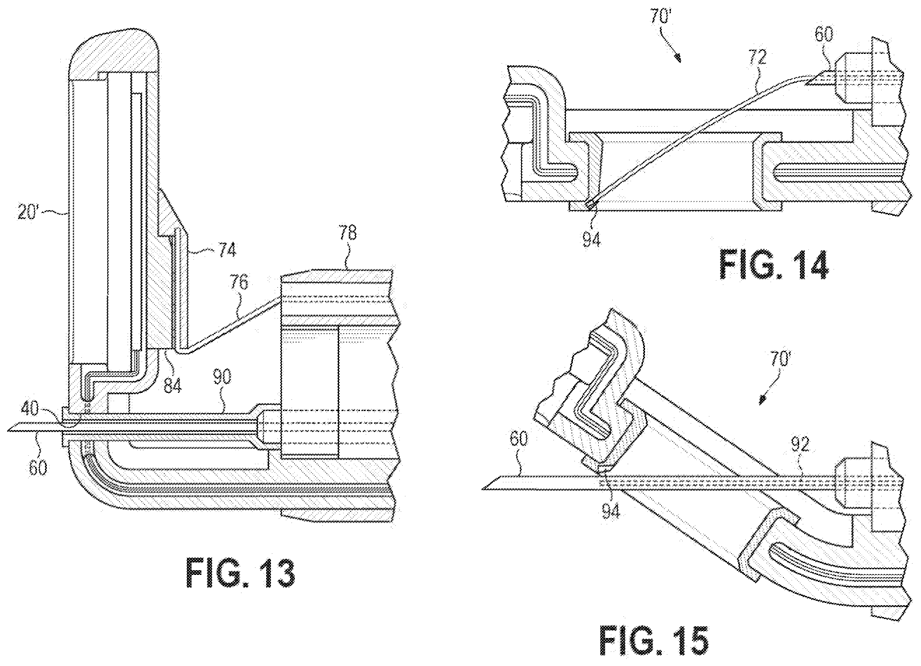

Another difference between assembly 70 and assembly 10 is the optional presence of a needle guide 90. FIG. 7 shows two additional variant needle guides 90′ and 90″. In assembly 10 it is possible that a needle 60 pushed out of a lumen of endoscope 12 could miss the aperture 40 in neck 38 and be blocked by neck 38 from further advancement. This might happen if a user attempted to push needle 60 into use when the neck 38 was not sufficiently pulled back, to bring aperture 40 into the correct position to let needle 60 pass through. The result could be damage caused to imaging head 20′, cause by needle 60. A needle guide 90 engages with aperture 40, so that needle 60 will be guided to aperture 40 with certainty, or will be blocked by guide 90, when head 20′ is not positioned correctly to align aperture 40 with the path of needle 60. FIG. 13 shows a needle guide 90 in use as head 20′ is pulled fully back, to a forward-looking position as needle 60 is advanced through aperture 40, with the assistance of guide 90. It is a further advantage of assembly 70 (and assembly 10) that the head 20′ can be moved to a forward-looking position as shown in FIG. 13, which is helpful to surgeons for some types of procedures. Referring to FIGS. 14 and 15, in a variant 70′ to assembly 70, a needle guide 92 is provided in the form of a wire that needle 60 advances over. When not in use, needle guide 92 is retained in a needle-guide notch 94 (FIG. 15).

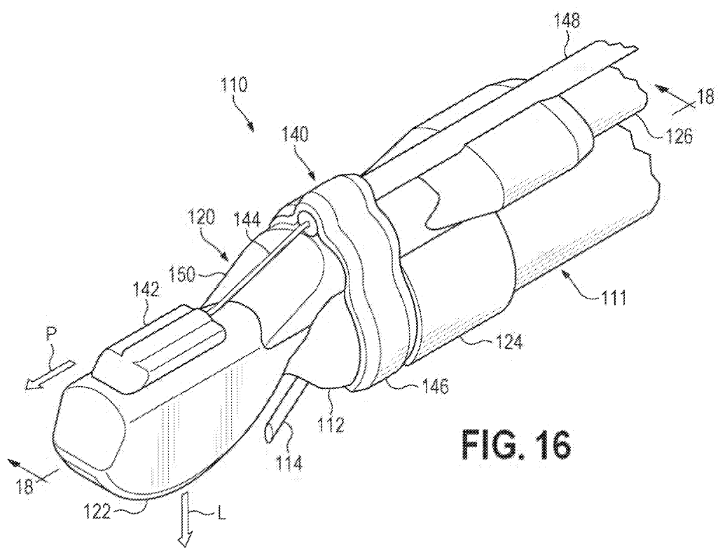

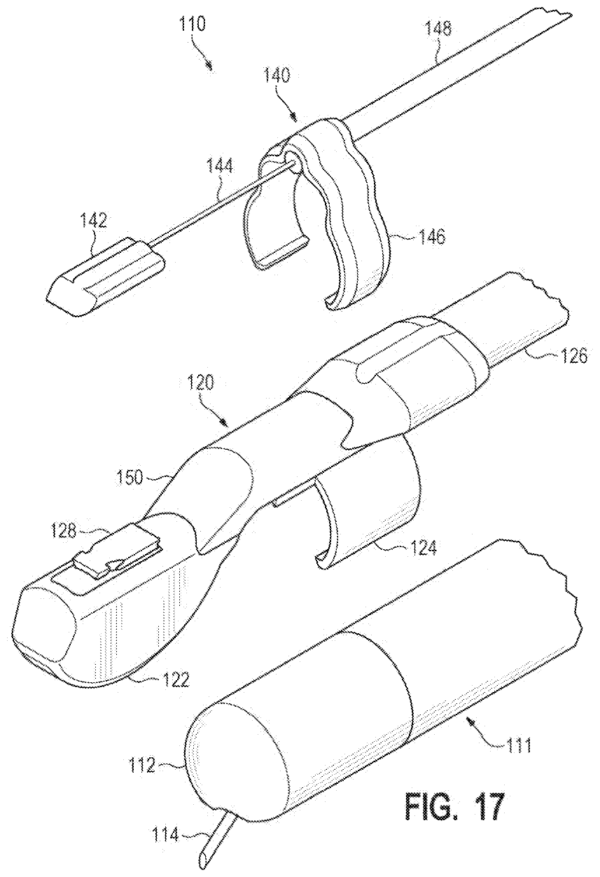

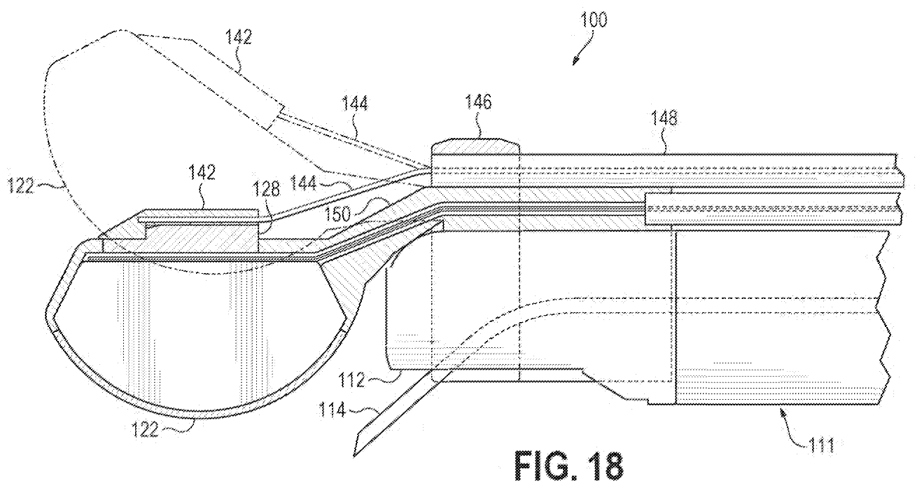

Referring now to FIGS. 16-18, a duodenoscope assembly 110 includes a duodenoscope 111 having a single-use instrument guidance head 112 (shown most clearly in FIG. 18), having an instrument guide 114 extending outwardly at an angle between a first lateral direction L and the distal direction P. Guidance head 112 can change the direction of guide 114, in response to varying user input via a tension member and an instrument variable guide member (not shown). Referring to FIG. 16, a cable/head sub-assembly 120 includes an ultrasound imaging head 122, a scope clip 124, a multiple signal pathway cable 126, delivering signals to imaging head 122 and relaying signals from imaging head 122. The signal pathways of cable 126 may be electrical conductors, and more specifically may each be a coax cable or a trace on a flex circuit. Other forms of signal pathways are possible. Imaging head 122 is shown having a signal emission surface facing the first lateral direction L, and a clip-hold 128 (FIG. 17) is present on head 122 on a side displaced from said signal emission surface in a second lateral direction, opposed to said first lateral direction L. An imaging head movement sub-assembly 140 includes a head clip 142, shaped to engage to clip-hold 128, a movement cable 144 a cable clip 146 and a conduit 148, holding the major portion of movement cable 144. Referring to FIG. 18 when cable 144 is pulled it pulls back imaging head 122 as indicated by the dotted line. In some embodiments sub-assembly 140 further includes an actuator (not shown) at the proximal end, to permit an operator to draw in cable 144, thereby pulling on imaging head 122 or let out cable 144, either pushing on imaging head 122 or permitting the resiliency of the material of cable 126 to place head 122 into a position more aligned with the longitudinal dimension of the duodenoscope 111, at its distal end. The actuator of cable 144 may take the form of a wheel, a lever or any other arrangement convenient to the user.

Because disinfection techniques typically require the application of chemicals in liquid form, thin crevices, into which liquid might not easily flow are generally undesirable. Accordingly, clip-hold 128 is designed so as not to define thin crevices with the imaging head 122. In alternative preferred embodiments, clip-hold 128 may have a shape that is similar to a knob, to further avoid defining any narrow crevices.

As noted in the background, the disinfection of devices such as assembly 110 is a matter of great concern, as there have been cases of the spread of strains of bacteria that are resistant to multiple antibiotics, by way of duodenoscope reuse. One area which may prove particularly difficult to sterilize is conduit 148, as movement cable 144 will tend to introduce body fluids into conduit 148 as cable 144 is pulled back into conduit 148, as imaging head 122 is moved back. To address this issue head movement sub-assembly is releasable and removable from the remainder of assembly 110 and is made to be inexpensive enough to use a single time and then be disposed. This eliminates the possibility of infection being spread from patient to patient by way of sub-assembly 140. Cable/head sub-assembly 120 does not have a similar structure that would provide a hard-to-reach place that would make disinfection difficult and will tend to be more expensive as it must contain a multiplicity of fine wires or other forms of signal pathways. Accordingly, cable/head sub-assembly 120 is designed to be cleaned and reused.

Before performing an endoscopic (duodenoscopic) procedure the endoscopist would obtain an unused head movement sub-assembly 140 and attach it to the remainder of assembly 110. After use, the user detaches and disposes sub-assembly 140.

Referring to FIG. 16, movement cable 144 may be pulled back to cause head 122 to face in a more distal facing direction. Pushing cable 144 forward causes head 122 to adopt a lateral viewing angle as shown, in one embodiment due to resilience of neck 150, but in another due to stiffness and compressive strength in cable 144.

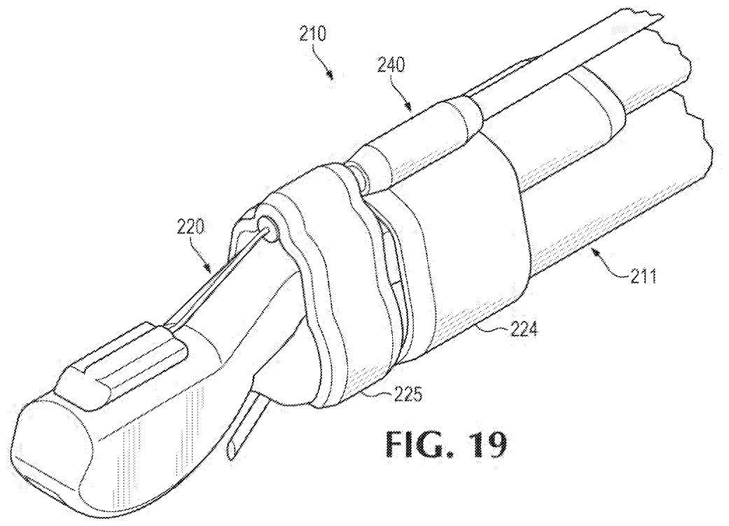

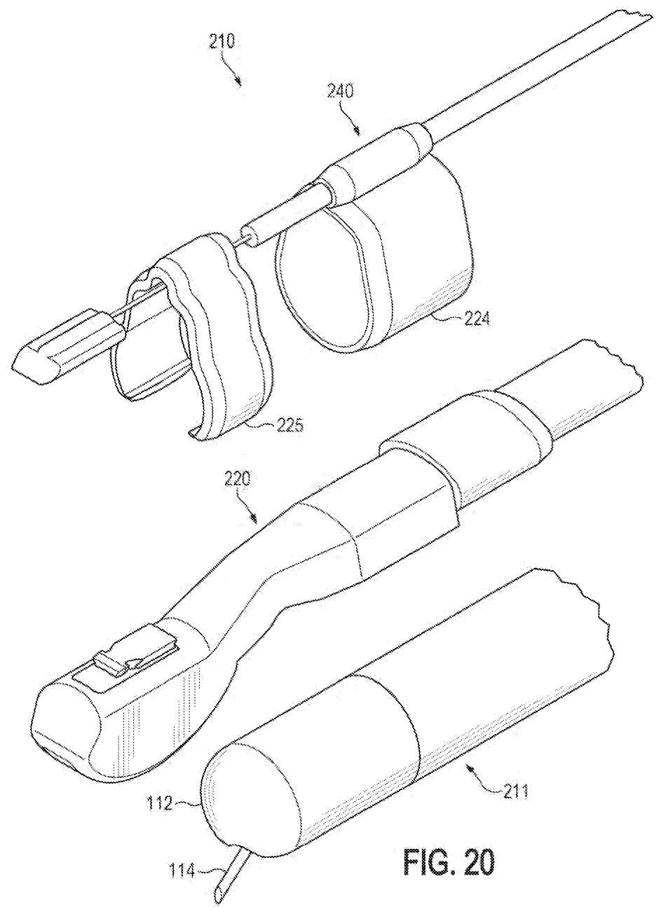

Referring to FIGS. 19 and 20, in an alternative preferred embodiment of a duodenoscope assembly 210, a holder 224 encompasses together both the duodenoscope 211, the cable/head sub-assembly 220 and the imaging head movement sub-assembly 240. A clip 225 also helps to hold the elements together.

In an additional set of embodiments and methods of use, any one of assembly 14 (combined with tension member 30), and assembly 70 and the combination of assemblies 120 and 140, can be made so that the resultant assembly 14/30, 70 or 120/140 (henceforth collective designated as assembly 14′) is produced and sold with a recommended method of use to dispose the entire assembly after a single use. This may greatly simplify health facility operations. In a preferred embodiment, the ultrasound array 52 (or the array in imaging head 20′ or 122) is a capacitive micromachined ultrasonic transducer (CMUT), which is generally less expensive than a piezoelectric transducer. Because cleansing an ultrasound assembly 14′ can be so cumbersome and expensive, and because of the great value of the surgeries being performed, even an assembly 14′ selling for upwards of $2,000 in 2019, could be more economical to dispose of, than to be cleansed and reused. In one embodiment of an assembly 14′, the number of array elements is reduced, from for example 256, to for example 128, or even to 64, to reduce the cost of the array, and the signal pathways leading to and from the array.

It shall be appreciated that the aforementioned ultrasound imaging sub-assemblies, ultrasound head movement assemblies, and needle guides can be used to enhance the utility of conventional endoscopes, duodenoscopes, and gastroscopes by providing selective modularity, disposability, and reusability. However, it shall be further appreciated that conventional needles may be too long for use with such assemblies, as they are designed for the conventional scopes, which are typically longer. Accordingly, there is a need for devices, systems, and methods adjusting the length of a conventional needle such that conventional needles can be used to be used in conjunction with the aforementioned ultrasound imaging sub-assemblies, ultrasound head movement assemblies, and needle guides to enhance the utility of conventional endoscopes, duodenoscopes, and gastroscopes.

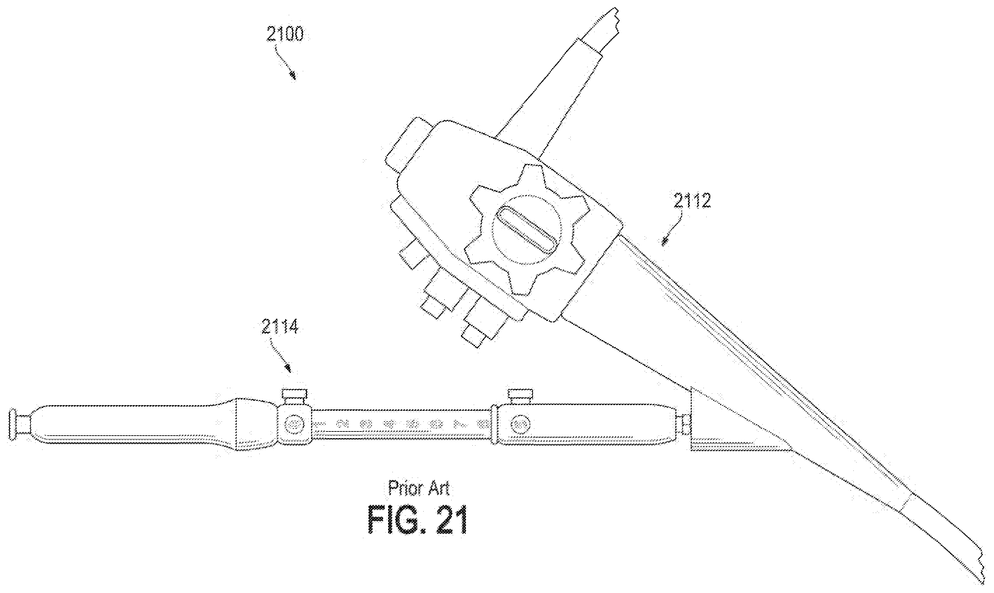

Referring to FIG. 21, an assembly 2100 is shown, including an endoscope 2112 (just the proximal end shown), and typical needle assembly 2114, attached to a proximal lumen port of the endoscope 2112, typically a “luer lock”. The only part of the needle assembly 2114 that is visible in FIG. 21 is the handle, which permits a surgeon to advance and retract the needle (or other device), which in the configuration shown, snakes through a lumen of endoscope 2112. For an endoscope 2112 that is an EUS scope, when the needle assembly 2114 is positioned as shown in FIG. 21, the distal end of the needle (not shown) will be located roughly at the distal end of the endoscope 2112, so that it can be extended out of the lumen and withdrawn into the lumen by manipulating the handle of assembly 2114. But, if endoscope 2112 were a gastroscope, the distal end of the needle would extend by a significant length, on the order of 2125 cm (10 inches) out of the distal end of the endoscope 12 (in this example a gastroscope), even when the handle of assembly 2114 was in it furthest retracted position. This would render the assembly unusable.

By virtue of a recent innovation, conventional gastroscopes can now function as EUS scopes. As noted, however, a conventional gastroscope is a different length then an EUS scope and therefore, when utilized as an EUS endoscope, the devices intended for various manipulations are not the correct length, but rather, are too long. There are other situations in which devices intended for a scope of one length are too long and need to be adapted to be used with a shorter endoscope.

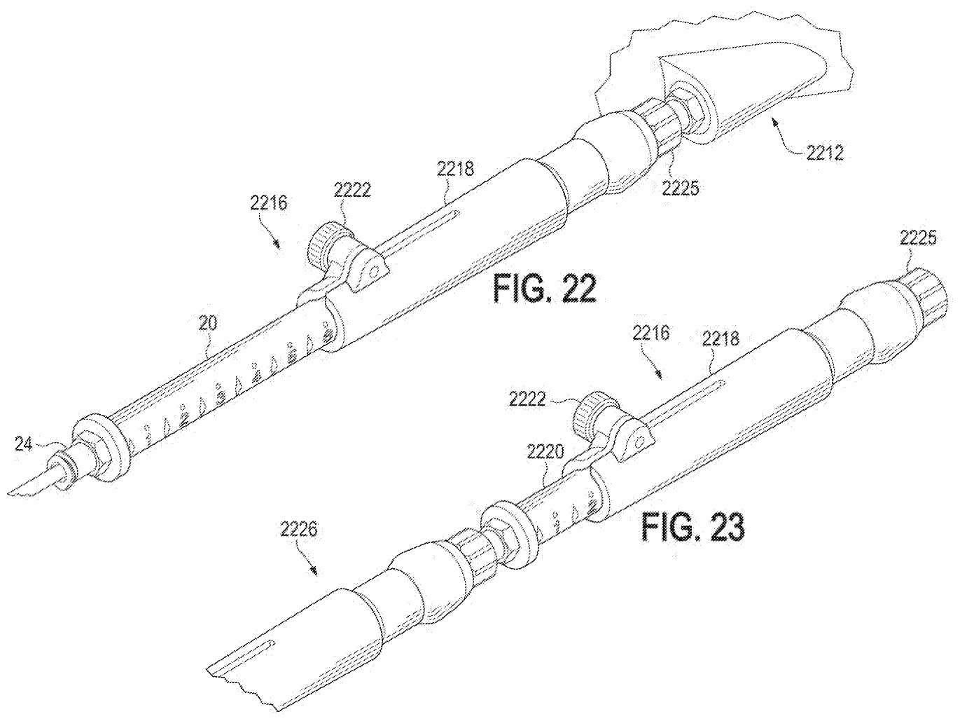

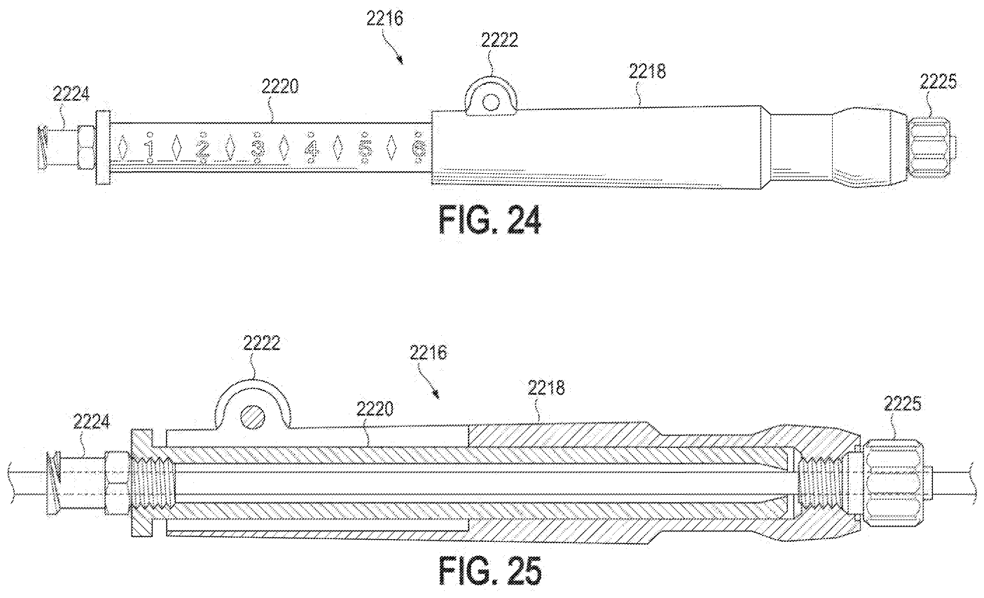

Referring now to FIGS. 22-25, in a preferred embodiment, an extender 2216 is provided and is attached (screwed into) to the proximal lumen port of endoscope 2212. An outer tube 2218 and inner tube 2220 telescope together to provide an adjustable length, and a gripping mechanism 2222 permits a user to fix the length of extender 2216 at a desired point. A distal threaded fitting 2224 is the same, on its insertive, distal end, as the insertive fitting at the proximal lumen port of endoscope 2212, thereby permitting whatever can attach to the lumen port of endoscope 2212, to attach to extender 2216. In like manner, extender 2216, at its distal end has a receptive connector 2225 for a proximal lumen port insertive connector, thereby permitting extender 2216 to connect to the proximal lumen port of endoscope 2212. Different endoscope makes (Pentax and Olympus) have different fittings for their proximal lumen ports. Accordingly, in one embodiment extender 2216 includes an adapter or set of adaptors (not shown) for permitting attachment to any make of gastroscope. Likewise, as each make of an EUS needle is adapted to fit a particular make of endoscope, extender 2216, in one embodiment, has an adapter or adapter set for connector 2225, so that any make of EUS needle can be accommodated. Referring more specifically to FIG. 23 in one configuration, another unit 2226 is attached to the proximal end of extender 2216. In one configuration, unit 2226 is an additional extender, but in another configuration, unit 2226 is a needle assembly, such as assembly 2214, but shown having a somewhat different distal shape.

Extender 2216 facilitates the use of standard EUS needles with endoscopes (such as gastroscopes) that are shorter than an EUS. The length adjustability supports use of such needles with a variety of endoscopes having varying lengths. Alternatively, an adjuster having a fixed length may be used.

The disclosed embodiments are illustrative, not restrictive. While specific configurations of the gastroscope proximal lumen port have been described, it is understood that the present invention can be applied to a wide variety of endoscopes. There are many alternative ways of implementing the invention. Additionally, it should be understood that in addition to biopsy needles, stents, radio-frequency ablation devices such as needles, cryotherapy devices such as needles, fiducial placement needles, and other such devices are often extended through the lumens of endoscopes, and accordingly the extender disclosed herein can be used with any of those devices.

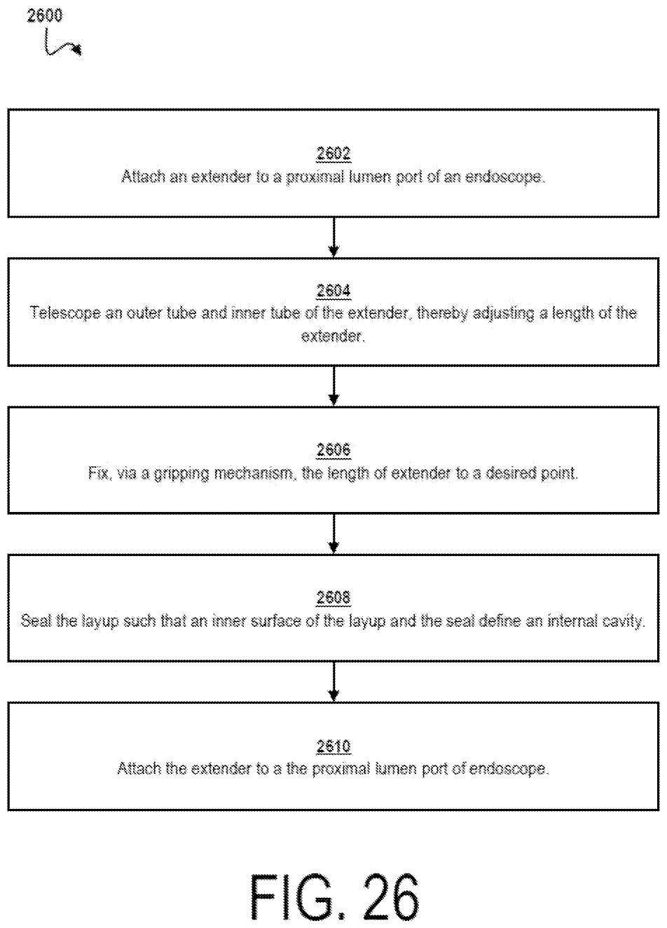

Referring now to FIG. 26, a flow chart illustrating a method of adjusting the length of a needle using the extender 2216 of FIG. 22 is depicted in accordance with at least one non-limiting aspect of the present disclosure. According to FIG. 26, the method 2600 can include attaching 2602 an extender 2216 (FIG. 22) to a proximal lumen port of an endoscope 2212 (FIG. 22). The method 2600 can further include telescoping 2604 an outer tube 2218 (FIG. 22) and inner tube 2220 (FIG. 22), thereby adjusting a length of the extender 2216 (FIG. 22). The method 2600 can further include fixing 2606, via a gripping mechanism 2222 (FIG. 22), the length of extender 2216 to a desired point. A user can then attach 2608 the extender 2216 (FIG. 22) to a the proximal lumen port of endoscope 2212 (FIG. 22). According to some non-limiting aspects, the method 2600 can further include adapting 2612, via an adapter, a connective port of the endoscope 2212 (FIG. 22) for use with an otherwise incompatible needle.

Various aspects of the subject matter described herein are set out in the following numbered clauses:
Clause 1: An endoscope assembly, including: an endoscope, having a proximal and a distal end, and defining a lumen, and having a proximal lumen port, permitting entry of a lengthwise needle or other device into said lumen; and a tubular extension, having a proximal and distal end, and having said distal end affixed to said proximal lumen port, an ultrasound endoscope needle assembly attached to proximal end of said tubular extension and having: a proximal handle; and a needle having a distal end, extending distally out of said handle; and wherein said needle fits through said tubular extension, and through said proximal lumen port into said lumen, and with said needle positioned so that said distal end is within said lumen but can be extended out of said lumen by manipulation of said handle.
Clause 2: The endoscope assembly according to clause 1, wherein said endoscope is a gastroscope.
Clause 3: The endoscope assembly according to clauses 1 or 2, wherein said tubular extension includes two tubes in telescoping arrangement, thereby permitting said tubular extension to be length adjusted.
Clause 4: The endoscope assembly according to any of clauses 1-3, wherein said tubular extension has a receptive endoscope proximal lumen port connector on said distal end, and an insertive endoscope proximal lumen port connector at said proximal end.
Clause 5: The endoscope assembly according to any of clauses 1-4, further including a first adapter positioned between said distal end of said tubular extension and said proximal lumen port.
Clause 6: A method of retrofitting an endoscope, having a proximal and distal end, and defining a lumen, and having a proximal lumen port, to accept and support an ultrasound endoscope needle assembly, having a handle that permits the extension and retraction of said needle, over a range, the method including: providing a tubular extension having a proximal and a distal end; affixing said distal end of said tubular extension to said proximal lumen port; and wherein said tubular extension is sized to permit said ultrasound endoscope needle assembly when affixed to said proximal end of said extension to extend through said extension and said lumen so that said distal tip of said needle can be withdrawn into said lumen and extended out of said lumen by manipulating said handle.
Clause 7: The method according to clause 6, wherein said endoscope is a gastroscope.
Clause 8. The method according to either of clauses 6 or 7, wherein said tubular extension includes two tubes in telescoping arrangement, thereby permitting said tubular extension to be length adjusted.
Clause 9: The method according to any of clauses 6-8, wherein said tubular extension has a receptive endoscope proximal lumen port connector on said distal end, and an insertive endoscope proximal lumen port connector at said proximal end.
Clause 10: The method according to any of clauses 6-9, wherein said step of affixing said distal end of said tubular extension to said proximal lumen port, includes interposing an adapter between said distal end and said proximal lumen port.
Clause 11: A tubular extension for a make of gastroscope, having a proximal and distal end, and wherein said distal end has a receptive connector for an endoscope proximal lumen port insertive connector for said make of gastroscope and wherein said proximal end has an endoscope proximal lumen port insertive connector.
Clause 12: The tubular extension of clause 11, wherein said tubular extension includes two tubes in telescoping arrangement, thereby permitting said tubular extension to be length adjusted.
Clause 13: The tubular extension of either of clauses 11 or 12, further including an adapter at its distal end, thereby forming said receptive connector for said endoscope proximal lumen port insertive connector for said make of gastroscope, by being fit onto a connector that would not mate with said endoscope proximal lumen port insertive connector.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations.” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect.” “in an aspect,” “in an exemplification.” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying.” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to.” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

1. A gastroscope conversion assembly, comprising:
a) a gastroscope, having a proximal and a distal end, and defining a lumen, and having a proximal lumen port, configured to permit entry of a lengthwise needle into said lumen, wherein said first needle comprises a first length; and
b) a tubular extension, having a proximal and distal end, and having said distal end affixed to said proximal lumen port;
c) an ultrasound endoscope needle assembly attached to proximal end of said tubular extension and having:
i, a proximal handle; and
ii, a needle having a distal end, extending distally out of said handle; and
d) wherein said needle of said ultrasound endoscope assembly comprises a diameter compatible with said lumen defined by said gastroscope and a length incompatible with said lumen defined by said gastroscope, and wherein said tubular extension can be adjusted to render said length of said needle of said ultrasound endoscope assembly compatible with lumen of said gastroscope, by enabling said needle of said ultrasound endoscope assembly to be positioned so that said distal end is within said lumen but can be extended out of said lumen by manipulation of said handle, thereby structurally converting said gastroscope for use as an EUS endoscope.

2. (canceled)

3. The endoscope assembly of claim 1, wherein said tubular extension is comprised of two tubes in telescoping arrangement, thereby permitting said tubular extension to be length adjusted.

4. The endoscope assembly of claim 1, wherein said tubular extension has a receptive endoscope proximal lumen port connector on said distal end, and an insertive endoscope proximal lumen port connector at said proximal end.

5. The endoscope assembly of claim 1, further including a first adapter positioned between said distal end of said tubular extension and said proximal lumen port.

6. A method of retrofitting an gastroscope, having a proximal and distal end, and defining a lumen, and having a proximal lumen port, to accept and support an ultrasound endoscope needle assembly, having a handle that permits the extension and retraction of said needle, over a range, the method comprising:
a) providing a tubular extension having a proximal and a distal end;
b) affixing said distal end of said tubular extension to said proximal lumen port; and
c) adjusting said tubular extension until a length of said needle of said ultrasound endoscope needle assembly is compatible with said lumen defined by said gastroscope, wherein said tubular extension is sized to permit said ultrasound endoscope needle assembly when affixed to said proximal end of said extension to extend through said extension and said lumen so that said distal tip of said needle can be withdrawn into said lumen and extended out of said lumen by manipulating said handle.

7. (canceled)

8. The method of claim 6, wherein said tubular extension is comprised of two tubes in telescoping arrangement, thereby permitting said tubular extension to be length adjusted.

9. The method of claim 6, wherein said tubular extension has a receptive endoscope proximal lumen port connector on said distal end, and an insertive endoscope proximal lumen port connector at said proximal end.

10. The method of claim 6, wherein said step of affixing said distal end of said tubular extension to said proximal lumen port, includes interposing an adapter between said distal end and said proximal lumen port.

11. A tubular extension for a make of gastroscope, having a proximal and distal end, and wherein said distal end has a receptive connector for an endoscope proximal lumen port insertive connector for said make of gastroscope and wherein said proximal end has an endoscope proximal lumen port insertive connector.

12. The tubular extension of claim 11, wherein said tubular extension is comprised of two tubes in telescoping arrangement, thereby permitting said tubular extension to be length adjusted.

13. The tubular extension of claim 11, further including an adapter at its distal end, thereby forming said receptive connector for said endoscope proximal lumen port insertive connector for said make of gastroscope, by being fit onto a connector that would not mate with said endoscope proximal lumen port insertive connector.
